(19) United States
(12) Patent Application Publication
(10) Pub. No.: US 2003/0180341 A1
(43) Pub. Date: Sep. 25, 2003

(54) Biocompatible hydrophilic films from polymeric mini-emulsions for application to skin

(76) Inventors: Jan W. Gooch, Atlanta, GA (US); F. Joseph Schork, Atlanta, GA (US); Albert T. McManus, Bulverde, TX (US)

Correspondence Address: SUTHERLAND ASBILL & BRENNAN LLP, 999 PEACHTREE STREET, N.E., ATLANTA, GA, 30309, US

(21) Appl. No.: 10/379,493

(22) Filed: Mar. 4, 2003

Related U.S. Application Data
(60) Provisional application No. 60/361,613, filed on Mar. 4, 2002.

(51) Int. Cl. A61K 6/00; A61K 31/155; A61K 33/14

(52) U.S. Cl. 424/401; 424/661; 514/634

(57) ABSTRACT

Polymer mini-emulsions are provided comprising a dispersed phase which comprises particles of a biocompatible hydrophilic polymer and having an average particle size between 50 and 750 nm, the polymer particles comprising a polymer of at least one ethylenically unsaturated monomer; an aqueous continuous phase in which said dispersed phase is dispersed; at least one co-stabilizer effective to stabilize the mini-emulsion; and optionally, a effective amount of at least one pharmaceutical agent, e.g., an anti-microbial agent. Biocompatible hydrophilic films formed from the mini-emulsions are provided, as are methods for making and using these barrier films on mammalian skin, particularly in the therapeutic treatment of serious skin wounds and burns. The flexible films tenaciously adhere to skin and are durable and washable. They preferably are permeable to air and water vapor, but can be formulated to substantially prevent gas or vapor permeation for use in contaminated environments.

### CROSS-REFERENCE TO RELATED APPLICATIONS

Priority is claimed under 35 U.S.C. §119 to U.S. provisional application Serial No. 60/361,613, filed Mar. 4, 2002.

### BACKGROUND OF THE INVENTION

This invention relates to topical materials for protecting and treating wounds in humans and other animals, and more particularly to polymeric materials for making protective films that adhere to wound sites.

The skin is the largest human organ and is the essential interface between man and his environment. The major functions of this organ include mechanisms for heat loss or heat retention, for water loss or water retention, for elimination of waste via exfoliation, for protection against penetration of ultraviolet light, for touch location of physical objects, and for protection of underlying tissues from microbial pathogens contacted in the environment. When the skin is damaged (e.g., by cuts, burns, or abrasions), it is desirable to provide an artificial barrier function over the damaged tissue, for example until the skin can heal itself or until surgical skin closure can be provided.

Methods to establish an artificial barrier function over damaged skin by the use of bandages, compresses, poultices, and other devices are well known throughout medical history. Today, adequate methods and devices are available for skin closure and/or bandaging at medical institutions capable of providing definitive surgical care (e.g., hospital emergency and operating suites). The general availability of such facilities and emergency transportation systems are basic infrastructural components of modern societies. The wide variety of sizes, shapes, materials, and mechanical devices necessary to accomplish this level of wound care, however, is dependent upon an extensive logistic and storage base.

In some situations, this extensive logistic and storage base, emergency transportation, and/or prompt surgical care are simply not available. For example, in a military setting, especially in the far forward stages of combat and other military use casualty care, such medical logistic assets are impractical and only become available in proportion to the increasing level of care provided at the various staging facilities of the evacuation process. Moreover, modern military conflicts often involve small operations (e.g., for antiterrorism campaigns, narrowly defined humanitarian/peacekeeping missions) with very limited external logistical support. Unlike previous major conflicts involving large numbers of combatants where logistical support is provided for rapid evacuation of wounded, the modern military operation may not be able to provide rapid or timely evacuation. Accordingly, injuries would be managed locally for longer post injury periods. This new time factor requires changes in front line medical capabilities, particularly with regard to wound care and prevention of infection.

Wounds suffered by military combatants often occur in soft tissues of the extremities. Soft tissues account for 44% of all casualties wounded in action. These wounds can range from life-threatening to trivial (although even a small infected wound can reduce one's operational effectiveness). The two conditions most greatly contributing to battlefield deaths are bleeding and infection, particularly from burns. Accordingly, it would be highly desirable to provide improved bandages and bandaging techniques for immediate treatment of bleeding from skin wounds, for immediate treatment and/or prophylaxis of infection in skin wounds, or both, particularly in settings where prompt surgery and/or other advanced treatments are not readily available. In particular, it would be highly desirable to provide an anti-microbial wound dressing that can be easily field applied to treat or prevent infection.

Conventional bandages and a number of other materials are available for use in covering topical wounds, such as cuts and burns. Many of these materials, however, do not actually provide the desired barrier function, particularly for an extended period of time, such as until the wound is healed, because they fail to provide an actual microbial barrier, are not durable, do not physically bond to human skin, need frequent replacement, or some combination of these shortcomings. It therefore would be desirable to provide a “super bandage” protective barrier for the protection of soft tissues, wherein the barrier is easy to apply, bonds to human skin, forms a protective barrier to environmental microorganisms, is air and water vapor permeable, but is washable and would remain on the skin for an extended period of time (e.g., until naturally sloughed off with dead skin cells). These features are particularly important to large area skin wounds such as found in burn patients. It would also be desirable if such a bandage were transparent or translucent to permit one to visually monitor the wound site and healing process.

One known alternate to conventional cloth or woven material bandages is the so-called “liquid” bandage. These liquid bandages typically have a main or base material comprising a cyanoacrylate or a nitrocellulose. For example, GluStitch™ (GluStitch, Inc., Vancouver, BC, Canada) uses N-2-butylcyanoacrylate, a monomer that polymerizes on contact with an aqueous fluid such as blood and becomes sticky. As another example, Dernabond™ utilizes 2-octyl cyanoacrylate, which also polymerizes on contact with aqueous fluids in/on the skin. Such products, however, typically do not possess long-term durability or washability after they are applied to the skin. Furthermore, when such products are applied to a wound leaking large amounts of fluid (e.g., blood, interstitial fluid, etc.), the curing process occurs only at the interface of the fluid and the product, resulting in no benefit to the tissue and subsequently flowing off the wounded area. In addition, they undesirably are applied in an organic solvent carrier, which may be irritating or painful upon application to a wound. Moreover, such products are only suitable for minor cuts and abrasions; they generally are unsuitable for serious wounds and burns, and are contraindicated for use on any wound with evidence of active infection or gangrene. It would therefore be desirable to provide an adherent protective covering that is suitable for serious wounds and that is effective in the treatment of infected or gangrenous wounds.

The patent literature discloses a variety of compositions purportedly useful as protective skin coverings. For example, U.S. Pat. No. 5,853,750 to Dietz et al. discloses polymerized micro-emulsion pressure-sensitive adhesives, which can be used as medical skin coverings and pharmaceutical delivery devices for application to mammalian skin. The bicontinuous micro-emulsions employ hydrophobic monomers in an organic phase and hydrophilic monomers in the aqueous phase, stabilized by large quantities of surface-active agents. It would be desirable to use another polymerization process rather than a micro-emulsion polymerization process. In addition, it would be desirable to provide films using lesser quantities of surfactants, as well as to provide emulsions that form better films, coatings.

As another example, U.S. Pat. No. 6,102,205 to Greff et al. discloses a prepolymer composition containing an antimicrobial agent complexed with a polymer for in situ formation of a polymeric film on mammalian skin. It would be desirable to provide a simpler system, which avoids in situ polymerization. Moreover, it would be desirable to avoid contacting a tissue wound site with monomers, toxic catalysts, and solvents, which would be present in such compositions prior to polymerization and which could react with tissues. Furthermore, such compositions would be unsuitable for treating effusive bleeding wounds and burns, particularly for patients in a hostile or severe environment.

In still another example, U.S. Pat. No. 6,139,856 to Kaminska et al. discloses an improved polyvinylidene fluoride-based composition for forming a substantially fluid resistant barrier film with skin adhesion properties. For use as a surgical drape, the film is dry, and organic solvent gone, before an incision is made. However, it would be undesirable to use the organic solvent system is wound dressing applications, as it would be desirable to avoid contacting a tissue wound site with irritating organic solvents.

In yet another example, PCT WO 02/34304 to Burnett, et al. discloses a self-adhesive, biocompatible and hydratable polymeric matrix, in the form of a sheet, patch, or film, which can be applied to the skin for use in wound healing. The matrix comprises a cross-linkable material that supports healing, such as albumin, and a synthetic bioadhesive polymer, and optionally a therapeutic agent. It would be desirable to provide a wound covering material that supports healing by protecting the wound with a non-chemically reactive barrier without interfering with tissue repair while allowing the presences of oxygen/water and the exodus of carbon dioxide.

It would be desirable to provide improved medical barrier materials and methods for easily applying such a protective barrier material over mammalian skin, particularly over severe tissue wounds (e.g., effusive bleeding cuts, burns, etc.). In addition, it would be desirable to provide a therapeutic barrier material useful in protecting or treating serious wounds, including those that are infected or gangrenous. Desirably, the protective barrier material would remain adhered to the skin until the wound is healed, or would serve as a triage covering to be removed by a surgeon once the patient is situated to receive professional medical care. Importantly, it would be advantageous for the barrier material to be biocompatible, to provide an actual barrier to microorganisms, to permit air and moisture to permeate through the barrier, and to be durable and washable.

### SUMMARY OF THE INVENTION

In one aspect, a polymer mini-emulsion is provided for forming biocompatible hydrophilic films for topical application to mammalian skin or soft tissues, particularly for use in the treatment of serious wounds, such as combat wounds and burns. The mini-emulsion comprises a dispersed phase which comprises particles of a biocompatible hydrophilic polymer and having an average particle size between 50 and 750 nm (e.g., between 100 and 500 nm), the polymer particles comprising a polymer of at least one ethylenically unsaturated monomer; an aqueous continuous phase in which said dispersed phase is dispersed; at least one co-stabilizer effective to stabilize the mini-emulsion; and, optionally and preferably, at least one pharmaceutical agent. In various embodiments, the pharmaceutical agent is selected from antimicrobial agents (e.g., chlorhexidine or a salt thereof), anesthetic agents, analgesic agents, hemostatic agents, and combinations thereof.

In one embodiment, the pharmaceutical agent is in the form of a finely divided solid dispersed directly in the aqueous continuous phase. In an alternative embodiment, the pharmaceutical agent is in a predispersion that is dispersed in the aqueous continuous phase.

In various embodiments, the ethylenically unsaturated monomer is selected from styrene, vinyl acetate, C1 through C4 alkyl acrylate, C1 through C4 alkyl methacrylate, butadiene, vinyl chloride, acrylic acid, methacrylic acid, and mixtures thereof. In a preferred embodiment, the polymer of at least one ethylenically unsaturated monomer comprises one or more acrylic polymers. In one embodiment, the dispersed phase comprises caprylic acid.

In one embodiment, the co-stabilizer comprises a monomer or polymer co-surfactant. The mini-emulsion also can comprise a hydrophobe, a plasticizer, or a combination thereof. In another embodiment, the mini-emulsion comprises a cross-linking agent effective to at least partially cross-link the polymer of at least one ethylenically unsaturated monomer.

In another aspect, a biocompatible hydrophilic film is provided for adhering to mammalian skin. Preferably, the film is a flexible, continuous film comprised of a biocompatible, hydrophilic polymer network formed from a mini-emulsion, as described above, wherein the film is permeable to air and water vapor and adherent to mammalian skin or soft tissue. In various embodiments, the biocompatible film is substantially transparent, substantially colorless, or both. In another embodiment, the biocompatible film comprises at least one water-dispersible pigment or dye in an amount effective to color the film. In one embodiment, the biocompatible film further includes a disposable substrate on which the film is maintained until transferred and adhered to the mammalian skin. In another embodiment, the biocompatible film further includes fibers dispersed therein, e.g., to provide additional film strength and/or abrasion resistance.

In a further aspect, a method is provided for forming polymeric film covering on mammalian skin. Preferably, the method includes the steps of (i) topically applying a liquid mini-emulsion, as described above, onto an area of human or other mammalian skin to form a wet layer; and (ii) drying the wet layer to form a flexible, continuous film adhered to the area of mammalian skin. In one embodiment, the area of skin comprises a serious skin wound, and the polymeric film covering provides a therapeutic effect. In one embodiment, the liquid mini-emulsion and the film formed therefrom comprise an anti-microbial agent in an amount effective to prevent or treat a bacterial infection in tissues in the skin wound. In other methods, the mini-emulsion and film comprise other pharmaceutical agents, alone or in combination with an anti-microbial agent. In one embodiment, the flexible, continuous film has an average thickness of between about 10 and about 25 microns.

In still another aspect, a method is provided for covering mammalian skin with an adherent polymeric film. The method comprises (i) providing a biocompatible, hydrophilic film which is formed from a liquid mini-emulsion, as described above; and (ii) contacting the biocompatible, hydrophilic film to an area of mammalian skin so that the film becomes adhered to the area of mammalian skin. In one embodiment, the biocompatible hydrophilic film has an average thickness between about 10 and about 50 microns.

In yet another aspect, a kit is provided for use in applying a biocompatible hydrophilic film to mammalian skin. Preferably, the kit of parts comprises (i) a container of a liquid mini-emulsion, as described above; and (ii) an applicator means capable of topically applying a layer of the liquid mini-emulsion onto an area of mammalian skin or soft tissues. For example, the applicator means may comprise a brush, a roller, a collection of woven or non-woven fibers, or a sprayer.

### BRIEF DESCRIPTION OF THE FIGURES

FIG. 1 is a cross-sectional view showing an area of skin on which a layer of the liquid polymeric mini-emulsion is dried to form a continuous biocompatible hydrophilic film.

FIG. 2 is a cross-sectional view showing a support substrate, a release coating on the substrate, and a biocompatible hydrophilic film on and then released from the release coating.

### DETAILED DESCRIPTION OF THE INVENTION

A biocompatible, hydrophilic acrylic polymer network film formed from a mini-emulsion has been developed, particularly for use in the treatment of severe tissue wounds (e.g., not scratches or other superficial wounds), such as combat wounds. The film can function as an adherent, protective skin covering, or barrier film. The film tenaciously adheres directly on skin, without the need for an accompanying adhesive, as the film utilizes significant hydrogen bonding. The barrier film is non-toxic and biocompatible, and optionally includes one or more pharmaceutical agents. Advantageously, the film is viscoelastic and compliant, i.e., it is capable of conforming to the tissue, flexing with the skin. It is durable, washable, and scrubbable. The barrier film preferably is applied to the skin as an aqueous mini-emulsion. The mini-emulsion will mix with blood and other bodily fluids immediately gelling the mix and forming a gelatinous film over the gelled fluid, which reduces bleeding and protects the underlying tissue. The mini-emulsion possesses very small particles that flow into the pores and crevices of the skin, where macro-emulsions and other conventional materials cannot. This feature importantly enables the mini-emulsion/film to anchor on the tissue. The mini-emulsion also is extremely stable to small particle size compared to other emulsions and materials.

In one aspect, the barrier film can shield the skin, particularly a wound therein, from contact by microorganisms and particulate dirt in/from the environment. The film preferably is permeable to air and water vapor, but can be formulated to substantially prevent gas or vapor permeation for use in contaminated environments. In one embodiment, the barrier film is semi-permeable, as it allows gases such as CO₂ to escape a skin wound, allows air and water vapor (e.g., perspiration) to permeate the healing tissue, and permits drainage of the wound. In an alternative embodiment, the barrier film can be formulated (e.g., greater crosslinking density, less water absorbance) to be impermeable to gas, vapor, chemicals, and microorganisms to thwart contact with biochemical weapons or other hazardous environmental agents, such as may exist in a combat or other hostile environment. Furthermore, the film can be transparent, which permits convenient visual inspection of the wound, to monitor the healing process without removing the covering.

As used herein, the term “polymer mini-emulsion” refers to a polymer emulsion (oil-in-water type) produced by polymerizing a monomer mini-emulsion under free radical polymerization conditions, wherein the average size of the monomer droplets is not in excess of 750 nm, and a co-stabilizer is included to stabilize the mini-emulsion.

As used herein, the term “biocompatible” means that the polymeric films do not elicit an undesirable biological response upon application to mammalian skin or skin wound therein.

As used herein, the term “hydrophilic” means that the polymer of particles and film possesses hydrophilic molecules, or hydrophilic groups of molecules, in sufficient quantity to interact with water present in skin to form a physical bond between the film and tissue.

The Mini-Emulsion and Film Compositions

The biocompatible hydrophilic barrier films are made from a stable polymer mini-emulsion, which is formed by polymerization of a monomer mini-emulsion. The polymer mini-emulsion comprises: a dispersed phase which comprises particles of a biocompatible hydrophilic polymer, the polymer particles comprising a polymer of at least one ethylenically unsaturated monomer; an aqueous continuous phase in which said dispersed phase is dispersed; and at least one co-stabilizer effective to stabilize the mini-emulsion. The mini-emulsion includes polymer particles that typically are substantially smaller than typical latex paints. The dispersed particles of the mini-emulsions have an average diameter between 50 and 750 nm. In one embodiment, the average diameter is greater than about 70 nm, and less than about 700 nm. In another embodiment, the average diameter is greater than about 100 nm (e.g., >125 nm, >150 nm, etc.) and less than about 500 nm (e.g., <400 nm, <250 nm, etc.). In a preferred embodiment, the composition includes one or more pharmaceutical agents.

The disperse or organic phase constitutes from about 10 to about 60 percent of total emulsion weight, and conversely the continuous or aqueous phase constitutes about 40 to about 90 percent of total emulsion weight. In one embodiment, the polymer mini-emulsion composition has a viscosity of about 500 cP. The viscosity can, however, be adjusted to essentially any value, as needed for various application purposes and methods.

The polymer mini-emulsion is made by a mini-emulsion polymerization process. Mini-emulsion polymerization begins with submicron droplets of monomer dispersed in an aqueous phase. High intensity fluid deformation and a co-surfactant are employed to generate and stabilize the small droplet size mini-emulsion. Particle nucleation is primarily via droplet penetration and, if most droplets are nucleated, the reagents are located at the polymerization sites and mass transport, except for the radicals, is not involved. Either water-soluble or oil-soluble initiators can be employed in mini-emulsion polymerization. Following polymerization, any residual monomers or catalyst can be steam stripped or chemically neutralized, preferably before addition of any pharmaceutical agents.

Mini-emulsion polymerization processes are described, for example, in U.S. Pat. No. 5,686,518 to Fontenot et al., U.S. Pat. No. 6,369,135 to Schork et al., U.S. Pat. No. 6,380,281 to Gooch et al., and U.S. Pat. No. 6,384,110 to Gooch et al., which are incorporated herein by reference. It should be noted, however, that the architectural coatings described in these references would generally not have the hydrophilicity necessary for use in the present biogel emulsions and films. In fact, hydrophilicity typically would be undesirable in such architectural coatings. Those methods are adapted for use in the present methods using biocompatible, hydrophobic polymers, particularly acrylic polymers and copolymers.

The mini-emulsions for forming biocompatible hydrophilic films preferably are essentially stable, to provide a sufficiently long shelf life of the mini-emulsion. For example, a container of the mini-emulsion should be able to be transported, stored, carried onto a battlefield to be ready for use several weeks, months, or even a year or more, after manufacture. The shelf life typically will vary depending on the amount and types of surfactants and co-stabilizers used.

Monomers and Co-Stabilizers

The monomeric formulation used to form the polymer particles can be a single monomer, or more preferably is a combination of two or more monomers selected to provide, for example, a favorable combination of cost, processability, stability, film forming properties, viscoelasticity, and hydrophilicity. In exemplary embodiments, the ethylenically unsaturated monomer is selected from styrene, vinyl acetate, C1 through C4 alkyl acrylate, C1 through C4 alkyl methacrylate, butadiene, vinyl chloride, acrylic acid, methacrylic acid, and mixtures thereof. This list is representative and not exhaustive. In a preferred embodiment, the polymer of at least one ethylenically unsaturated monomer comprises one or more acrylic polymers. As used herein, the terms “polymer,” “polymers,” or “polymeric” are used broadly to include homopolymers, copolymers, block or graft copolymers, or blends or mixtures thereof, unless otherwise explicitly indicated.

The mini-emulsion requires at least one co-stabilizer in an amount effective to stabilize the mini-emulsion. The co-stabilizer is sometimes referred to as co-surfactant. The co-stabilizer is added to the monomer emulsion to stabilize the monomer droplets against diffusional degradation. It must be extremely soluble in the monomer (or monomer mix) and extremely insoluble in the aqueous phase. It may or may not function as a reactant in the monomer polymerization, depending upon the co-stabilizer selected. The co-stabilizer can be polymeric or non-polymeric. In various embodiments, a single non-reactive cosurfactant is used, or a mixture of polymeric, non-polymeric, or polymer and non-polymeric cosurfactants is used.

In a preferred embodiment, the co-stabilizer is a monomer that is later polymerized into the polymeric matrix, or is a polymer. In a preferred embodiment, the co-stabilizer is the co-monomer dioctyl maleate. A polymeric cosurfactant is a polymer that is both highly water insoluble and highly soluble in the monomer of choice. The polymeric cosurfactant may be a polymer or a mixture or blend thereof having a molecular weight in the range of about 3,000 to about 1,100,000, preferably from about 9,000 to about 750,000. Especially preferred non-reactive polymeric cosurfactants are those having a molecular weight in the range of about 350,000 to about 750,000. Representative polymeric cosurfactants suitable for use herein include polymethyl methacrylate (PMMA), polystyrene, polyvinyl acetate, polymethylacrylate and polyethylacrylate. Certain copolymers such as styrene-isoprene copolymer, and certain block polymers such as poly(styrene-block-butadiene) and poly(styrene-block-isoprene) are also useful. Other polymeric cosurfactants may be used, if they are essentially insoluble in water but soluble in the monomer or monomer mixture.

In one embodiment, the polymeric non-reactive cosurfactant is a polymer of the monomer undergoing polymerization. For example, polymethyl methacrylate is a preferred non-reactive cosurfactant when methylmethacrylate is the monomer. In other embodiments, the polymeric non-reactive cosurfactant is a polymer that is not the polymer obtained by polymerization of the monomer of choice.

The amount of polymeric cosurfactant generally is between about 0.5 and about 5.0 percent by weight (wt %) based on the monomers and the polymeric cosurfactants.

In other embodiments, non-polymeric cosurfactants are used, either in place of or in combination with polymeric cosurfactants. Representative examples of nonpolymeric non-reactive cosurfactants include hexadecane and cetyl alcohol. Non-reactive nonpolymeric cosurfactants, when used, are added in a concentration between about 0.5 and about 5 percent by weight (wt %) based on the monomers and cosurfactants.

Conventional surfactants for emulsion polymerization also generally must be included, either as a single surfactant or a mixture of surfactants. Representative examples of such surfactants include sodium lauryl sulfate and other alkyl sulfates, sodium dodecyl benzene sulfonate and other alkyl and aryl sulfonates, sodium stearate and other fatty acid salts, and polyvinyl alcohol and other non-ionic surfactants. The surfactant may be either an anionic, cationic or a non-ionic surfactant. When a mixture or combination of surfactants is used, the mixture may include an anionic or a cationic surfactant, plus a non-ionic surfactant, or two or more anionic or cationic surfactants, or two or more non-ionic surfactants. The amount of surfactant is from about 0.5 to about 5.0 percent by weight, based on monomers.

One exemplary formulation is described below in Example 1. Those skilled in the art can readily derive other suitable variations and formulations.

Pharmaceutical Agents and Excipients

In a preferred embodiment, the biocompatible hydrophilic films include one or more pharmaceutical agents, for use in a variety of therapeutic or prophylactic applications. Typically, the pharmaceutical agent is controllably released from the film to the soft tissue over which the film is applied. The pharmaceutical agent can be released for local, regional, or systemic effect.

In a preferred embodiment where the film is used as a bandage to cover a wound, the film includes an effective amount of one or more pharmaceutical agents. As used herein, the term “pharmaceutical agent” includes therapeutic and prophylactic agents. An agent may function both as a therapeutic and a prophylactic.

For example, the pharmaceutical agent could be selected from antimicrobial agents, anesthetic agents, analgesic agents, hemostatic agents, and combinations thereof. A wide variety of other pharmaceutical agents known in the art could be used, depending upon the intended use of the biocompatible films.

In one embodiment, the pharmaceutical agent is an anti-microbial agent and the film is to be topically applied to mammalian skin, particularly over a skin wound. Representative anti-microbial agents include biguanides, especially chlorhexidine and its salts, including chlorhexidine acetate, chlorhexidine gluconate, chlorhexidine hydrochloride, and chlorhexidine sulfate; silver and its salts, including silver acetate, silver benzoate, silver carbonate, silver iodate, silver iodide, silver lactate, silver laurate, silver nitrate, silver oxide, silver palmitate, silver protein, and silver sulfadiazine; polymyxin; tetracyclines such as tetracycline hydrochloride, doxycycline hyclate, and minocycline hydrochloride, azithromycin, and clarithromycin; aminoglycosides, such as tobramycin and gentamicin; rifampicin; bacitracin; neomycin; chloramphenicol; miconazole; quinolones such as oxolinic acid, norfloxacin, nalidixic acid, pefloxacin, enoxacin and ciprofloxacin; penicillins such as oxacillin and pipracil; nonoxynol 9; fusidic acid; cephalosporins such as ceftazidime; and combinations thereof.

Examples of preferred anti-microbial agents include chlorhexidine diacetate, chlorhexidine glutonate, 4-(aminomethyl)benzenesulfonamide hydrochloride hydrate, P-(aminomethyl)benzenesulfonamide hydrochloride, and P-(aminomethyl)benzenesulfonamide acetate salt.

In one preferred embodiment, the anti-microbial agent is sodium hypochlorite, which was effective as a broad spectrum anti-microbial when provided in the mini-emulsion/film compositions, preferably in an amount between about 0.1 and about 0.5% by weight of solids.

Non-limiting examples of other classes and species of pharmaceutical agents that may be useful in the compositions, films, and methods of uses described herein include analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, and meprobamate); antifungal agents (e.g., griseofulvin, ketoconazole, itraconizole, virconazole, amphotericin B, nystatin, and candicidin); anti-inflammatories (e.g., (non-steroidal) celecoxib, rofecoxib, indomethacin, ketoprofen, flurbiprofen, naproxen, ibuprofen, ramifenazone, piroxicam, (steroidal) cortisone, dexamethasone, fluazacort, hydrocortisone, prednisolone, and prednisone); antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, clarithromycin and colistin sulfate); and antiviral agents (e.g., interferons, zidovudine, amantadine hydrochloride, ribavirin, and acyclovir).

The polymeric films also may include one or more excipient materials. The term “excipient” refers to any non-active ingredient of the formulation intended to facilitate controlled release and topical delivery of the pharmaceutical agent. For example, the excipient can comprise sugars or other carbohydrates, starches, lipids, or combinations thereof. The excipient may enhance handling, stability, and dispersibility of the active agent (e.g., the therapeutic agent). Examples of excipients include pharmaceutically acceptable carriers and bulking agents, osmotic agents, binders, disintegrants, diluents, color agents, and lubricants.

The pharmaceutical agent can be incorporated into the film in varying amounts, depending upon, for example, the particular medical application, the type of wound, the type or location of the soft tissue, the selection of pharmaceutical agent, and the amount of agent required to achieve the desired bioactive effect (i.e., a pharmaceutically effective amount). Other factors include the need to provide a stable mini-emulsion and sufficient hydrophilic properties of the film. In one embodiment, the mini-emulsion comprises an anti-microbial agent in an amount greater than about 0.1 wt % (e.g., >0.2 wt %, >0.5 wt %, >1.0 wt %, etc.) and less than about 50 wt % (e.g., <40 wt %, <20 wt %, <15 wt %, etc). In a preferred embodiment, the mini-emulsion comprises a chlorhexidine salt in an amount between 0.5 and 10 wt %.

The pharmaceutical agent can be added directly as a solid or liquid (pure or solution) to the aqueous phase of the emulsion. In embodiments where the pharmaceutical agent is insoluble in the aqueous phase, the pharmaceutical agent preferably is in very finely divided form, e.g., by micronization, grinding, milling, or the like, or is dissolved in the monomer droplets. A particle size of less than about 20 μm is generally desirable for solid pharmaceutical agents. In one embodiment, the finely divided solid form of pharmaceutical agent is added by slowly stirring the emulsion at room temperature (or below) without attempting to dissolve the solid agent. This procedure would produce a solid powder within a liquid emulsion type dispersion. Care should be taken to maintain the stability of the emulsion when adding any type or amount of pharmaceutical agent or excipient material by monitoring the ionic strength in the aqueous phase, pH, and other process parameters. Additional surfactant and/or pH adjustment can be used as needed to facilitate blending and stability of the pharmaceutical agent and/or the polymer film. If emulsion destabilization can be avoided, direct addition of pharmaceutical agent to the aqueous phase is desirable for pharmaceutical agents requiring direct contact with or immediate release into the skin or other soft tissues (in contrast to the delayed release or indirect contact provided when the pharmaceutical agent is predispersed, as described below).

In another method, the pharmaceutical agent is not added the aqueous phase directly, but is first predispersed in one or more other liquids (e.g., a hydrophobic medium), in order to avoid destabilizing the mini-emulsion due to the agent's ionic activity. For example, the pharmaceutical agent can be dispersed in an oil first and then in a surfactant prior to addition to the mini-emulsion. Examples of these biocompatible oils include vegetable oils. Alternatively, the pharmaceutical agent could be dissolved in the monomer phase before the mini-emulsion is made. It is noted, however, that the rate or release of the agent from the predispersion or hydrophobic medium typically is slower than the agent would be from the pure, exposed agent. Predispersion in a biocompatible oil is sufficient to coat the particles and shield them from ionic activity. Additional biocompatible surfactant can be added to disperse the predispersion in the aqueous phase of the mini-emulsion.

In a typical recipe, the amount of pharmaceutical agent to be added into the mini-emulsion is first determined, and then the amounts of dispersant and surfactant needed to prepare the predispersion can be determined based on weight percent of the pharmaceutical agent. For example, in one formulation of an anti-microbial agent predispersion, the weight percentages of anti-microbial agent, dispersant (oil or other liquid hydrophobe), and surfactant (alkylaryl polyether alcohol) are 24.5, 69.9, and 5.6, respectively.

In one embodiment, caprylic fatty acid is formulated into the polymer phase to serve as a biocompatible plasticizer for the polymer phase and subsequent dried films. These films and mini-emulsions containing caprylic fatty acid were found to provide antifungal and antibacterial properties. In particular, the dried film exhibits broad spectrum antifungal properties and provides a mild antibacterial effect. The liquid mini-emulsion, however, exhibits broad spectrum antibacterial properties.

Other Additives

Various additives can be included in the mini-emulsion to enhance one or more properties of the barrier films formed from the mini-emulsion, and/or one or more physical properties of the mini-emulsion.

In one embodiment, fine, biocompatible fibers can be added to the mini-emulsion to provide the dried film with randomly oriented fibers lying in the plane of the dried film, in order to impart additional strength to the film. The fibers can be, for example, highly purified short cellulose fibers or other biocompatible fibers. They can be added to the emulsion in a pre-dispersion of water and surfactant. The amount of the fibers in the film can vary; however, it was found that between about 0.5 and 1.0 wt % of solid fibers in the emulsion will produce a tougher and more abrasion resistant film.

In another embodiment, the mini-emulsion includes one or more cross-linking agents effective to cross-link, at least partially, the polymeric materials. The cross-linking agent can be used to improve the physical properties of the film, for example, to render the film less gooey or sticky. An exemplary cross-linking agent is divinylbenzene, although a variety of others may be suitable.

Other additives are used to control the hydrophilicity and solubility in water of the mini-emulsion particles, for example to improve the film formation and enhance the flexibility of the biocompatible films. In one embodiment, these properties are controlled by manipulating the concentration of vinyl acetate monomer in the monomeric formulation. Polymerized vinyl acetate is dispersible (e.g., swells) in water, but is not soluble therein. Therefore, the tackiness, which is related to hydrophilicity, of the particles can be controlled by the concentration of the vinyl acetate polymer segment in the resultant copolymer. For example, to increase hydrophilicity and tackiness, one could decrease the concentration of vinyl acetate monomer. Experimental data shows that the relationship between percent weight vinyl acetate and surface tackiness is roughly linear by weight (but not by mole fraction). As the amount of hydrophobe that can be added to the monomer mixture before polymerization is limited, a plasticizer, such as a benzoate ester, including diethylene glycol dibenzoate/dipropylene glycol monobenzoate or a mixture of both, is preferred. Plasticizers other than benzoates can also be used. The freezing temperature of the solids in the emulsion can be lowered by adding a “lower temperature with lower viscosity” plasticizer. The freezing temperature of the emulsion can be lowered by adding ethylene glycol or another lower temperature solvent to the aqueous phase.

The thickness of a film can be adjusted simply by controlling the quantity of liquid mini-emulsion applied to a particular area of substrate or skin. The tensile strength and viscoelastic properties of the film can be controlled by the composition of monomers that are polymerized to form the polymeric particles in the mini-emulsion.

Polyvinyl alcohol can be added to increase the tack and hydrophilicity and to cause the mini-emulsion to gel, and form a film, on contact with moisture in a wound.

Applications and Methods of use

The biocompatible, hydrophilic films have a variety of medical and non-medical uses, particularly as adherent skin coverings for several different purposes. The barrier film can be applied to the skin as an aqueous mini-emulsion or as a pre-formed film (made from an aqueous mini-emulsion).

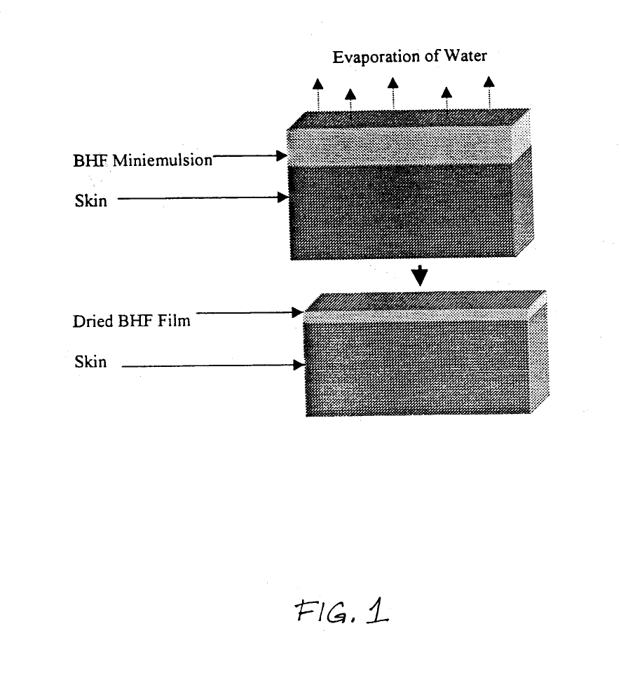

In the first case, the mini-emulsion can conform to essentially any skin area and then dry into a thin film (see FIG. 1). In one embodiment, the dry film has a thickness greater than about 10 microns (e.g., >15 μm, >20 μm, etc.) and less than about 50 microns (e.g., <40 μm, <30 μm, <25 μm, etc.). The liquid mini-emulsion can be applied to an area of mammalian skin (or an intermediate drying substrate) by a variety of liquid applicator means, including, but not limited to, the use of a brush, swab, roller, or spraying device. Following application of the mini-emulsion onto the desired surface, the aqueous phase of the mini-emulsion is evaporated to leave behind a flexible, polymeric, continuous membrane (i.e., a stable, semi-permeable film) from the dispersed phase of the mini-emulsion.

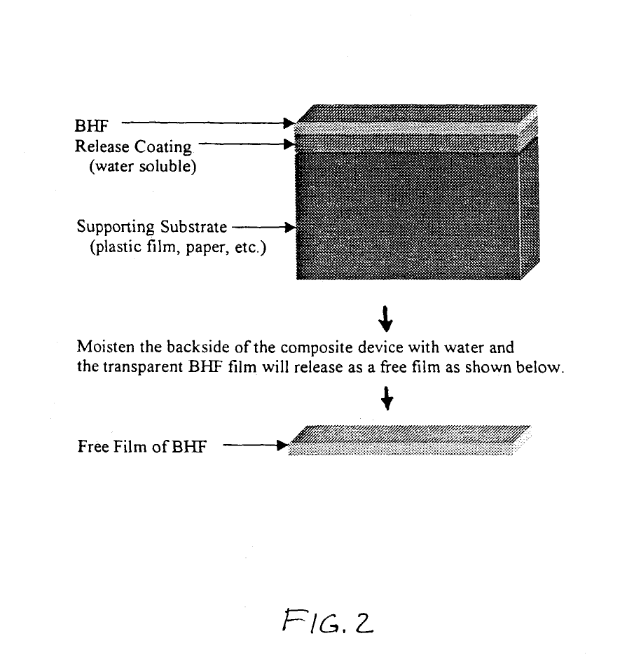

In the second case, strips or bandages of the pre-fabricated barrier films can be draped or wrapped over an area of the skin. The barrier film can be pre-fabricated by applying the mini-emulsion to a support substrate, drying it for packaging, distribution, storage, and use. Alternatively, the film may be transferred to a secondary substrate following the drying step. The support substrate (e.g., paper or plastic film) can be provided with a hydrophobic coating, or water soluble release coating, to facilitate removal of the hydrophilic films. To use the film, the backside of the composite structure could be moistened with water, causing the barrier film to release as a free film for transfer to skin (see FIG. 2).

The adhesion properties of the biocompatible hydrophilic films are provided by the hydrophilic attraction of the hydrophilic polymers to moisture in the skin. Strong hydrogen bonding and other secondary forces (polar and dispersion) prevent removal of the film. Hydrogen bonding is especially effective between soft tissues and a surface covered with hydroxyl and carboxyl groups. In addition, as gases and water vapor diffuse from the skin and through the film adhered on the skin, a partial vacuum is created at the interface between the film and the skin, while atmospheric pressure exerts a force on the planar surface of the thin, pliable film. This restricts movement of the film. The films can remain adhered to the skin in excess of two weeks, when it will slough off naturally with dead skin cells, or it can be removed earlier by using an alcohol solvent. In one embodiment, the film can serve as a triage covering to be removed by a surgeon once the patient is situated to receive professional medical care.

The films can be mass produced and packaged for individual use as needed. Alternatively, mini-emulsions for forming the films can be packaged and stored for use as needed, for example in a kit form as detailed below. In either case, the packaging preferably is designed to promote or maintain the sterility of the film or mini-emulsion, particularly if it is intended for wound treatment applications.

In one aspect, a kit can be provided, which includes a container of the mini-emulsion and an applicator means that can be used to topically apply the mini-emulsion to the skin. For example, the applicator means and container could be combined, e.g., in the form of a bottle or can having a spray nozzle attached thereto for spraying the mini-emulsion contained inside the bottle or can. The spray nozzle could operate under pressure, e.g., with a propellant, or it could be a conventional pump type. In another embodiment, the container has a conventional closure and the applicator means comprises a brush, a roller, and/or a collection of woven or non-woven fibers (e.g., a swatch of cotton gauze, a cotton swab, and the like).

Wound Treatment

A particularly advantageous application for the biocompatible hydrophilic films is in the treatment and protection of wounds to mammalian skin. In particular, the films serve as a protective barrier for skin cuts and abrasions, burned skin tissue, and other external soft tissue wounds. The films can include one or more pharmaceutical agents to further aid in the prevention or treatment of wound infection, to reduce pain or inflammation, and to otherwise aid in the healing process. The mini-emulsion can be topically applied to a patient's wound (e.g., sprayed, rolled, or wiped over the wound site to cover the wound). The mini-emulsion then is allowed to dry to form the barrier film-which acts as a second or replacement skin. In one embodiment, the dried film has an average thickness of between about 10 and 25 microns. In a preferred embodiment, the barrier film is substantially colorless and substantially transparent, e.g., enough to permit one to visually monitor the wound site without removing the protective barrier.

In another embodiment, the film is formed before it is applied to the wound. For example, the emulsion can be applied to a substrate, e.g., the surface of a mold, a sterile planar surface, or the like. The mini-emulsion would then be allowed to dry and form the film. The film would then be removed for use. The film could be packaged and stored for later use. The film could be packaged on the substrate it was dried on, transferred to another substrate or carrier, or packaged without any substrate. In one embodiment, a large sheet is formed, and then cut into smaller sections suitable for use. This could be done in a continuous process, e.g., for large scale manufacturing.

In a preferred embodiment, the mini-emulsion includes a pharmaceutical agent that aids in the healing process. For example, the pharmaceutical agent could be selected from anti-microbial agents, anesthetic agents, analgesic agents, hemostatic agents, and combinations thereof. In one embodiment, the mini-emulsion includes an anti-microbial agent dispersed therein in an amount effective to treat or prevent a bacterial infection. Chlorhexidine is particularly suitable, as it is soluble is warm water and will dissolve when it exposed to the moisture in the skin (at about 37° C.). It therefore can remain a solid trapped in the mini-emulsion or film until it is applied, and then it can dissolve and diffuse out of the film in contact with wounded skin tissues.

The film desirably is durable and water insoluble, permeable to air and water (except as otherwise noted herein), but effectively impermeable to micro-organisms. In a preferred embodiment, the film is transparent or translucent, so that the wound can be examined during the healing process without having to remove the film. The film, however, generally is safely removable with an alcohol (e.g., isopropyl alcohol, ethanol, etc.) or another organic solvent that is safe for topical use. The film could be removed if a surgeon or other medical professional needs to access to the wound (e.g., following triage application) or when the wound has healed sufficiently to avoid or eliminate infection. Alternatively, the film is sloughed off naturally with the skin

The biocompatible hydrophilic films are particularly desirable for use in situations when and where immediate access to medical equipment and/or medical personnel is not available. For example, the mini-emulsion and film compositions described herein would be particularly useful in the treatment of combat soldiers or other military personnel in the field. The soldiers' wounds could be covered with the films to treat or prevent wound infections. This treatment of wound infections could be a very important application, since several conventional materials are contraindicated for use on any wound with evidence of active infection or gangrene. The mini-emulsion and film compositions described herein also would be useful in treating civilian victims of disasters, war or terrorist attacks, or burn victims.

In yet another embodiment, the emulsions and films could be used in the treatment of a baby's diaper rash. The film could be prevent urine and feces from contacting the baby's skin at the site of the rash, and the washability of the film would be highly useful to keeping the area clean.

In another embodiment, the emulsions and films can be used in veterinary applications. For example, the emulsions and films can be used to treat skin wounds in dogs, cats, horses, cattle, or sheep. It also could be used to treat skin wounds in various zoo animals, including monkeys and other mammals.

Protective Barrier for Healthy Skin

Another application is to provide a protective, artificial skin for the prevention of or resistance to skin abrasions and cuts and blisters. In one embodiment, an athlete could apply the film to skin areas that are exposed or otherwise prone to incur incidental abrasions, cuts, blisters, and the like, when the athlete participates in various sports and activities. For example, a soccer player could apply a layer of the barrier film to shins and knees to shield against bleeding or abrasions from contact with the turf or others' cleats. Similarly, a runner could apply a layer of the barrier film to his feet or a tennis player could apply a layer to areas of the fingers and hands to prevent or reduce the formation of blisters. In these types of barrier applications, relatively thicker film layers may be preferred, and the films may desirably include randomly oriented fibers to provide enhanced film durability and/or rigidity.

In yet another application, the barrier films can be used for the shielding the skin from contact with irritating or harmful materials. For example, a film could be applied onto to one's feet prior to wading through swampy water wearing boots to prevent contact with microbes, such as fungi. This could be particularly useful to soldiers, campers, hunters, field researchers, and others in need of such protection. In another example, the film could be applied to the hands of one who needs to handle, or may inadvertently contact, a harmful or irritating fluid, such as in an industrial workplace. Aircraft mechanics, for example, often have their hands exposed to aircraft fluids, including polyester hydraulic fluids, that are irritating to exposed skin. Application of the barrier films to the hands prior to exposure could prevent or reduce skin irritation.

In another aspect of protecting healthy skin, the mini-emulsion and film compositions could include an effective amount of a chemical sunscreen, an insect repellant, or a combination thereof.

Drug Delivery

The wound treatment methods could be applied to healthy skin tissues for transdermal drug delivery applications. For example, small molecule drugs could be readily incorporated into the mini-emulsion for transdermal systemic or local administration. Alternatively, a nicotine patch, for use in smoking cessation therapy, could be pre-fabricated from the adherent films described herein. Those skilled in the art could adapt the films for use in other therapeutic or prophylactic transdermal drug delivery applications.

Cosmetic and Aesthetic Applications

The biocompatible hydrophilic films also can be used in a variety of non-medical applications. Examples of such non-medical applications for the films include skin decals, temporary “tattoos”, sunscreens, and camouflage for the skin, as well as cosmetic or prosthetic devices for application to the skin.

For example, the films could be provided with coloring, designs, letters, or symbols, such as for functional or artistic purposes. Such elements could be added to the film before, during, or after film formation, or in some combination thereof, depending on the materials being incorporated. Colored films can be provided, for example, by coloring the mini-emulsion using water dispersible dyes and pigments. Alternatively or in addition, the barrier film can be provided with an overlayer (continuous or discontinuous) that includes various coloring, patterns, designs, symbols, etc.

As another example, liquid-applied clear or skin color pigmented dried films could be provided to cover skin blemishes. Such materials could be adapted to be a very natural looking cover, which is long lasting and waterproof. The natural, translucent effect from this material can be pigmented for individual color-tone to provide a perfectly matching surface that blends with natural skin tones.

The present invention can best be understood with reference to the following non-limiting examples.

### EXAMPLE 1

### Production of a Hydrophilic Skin Barrier Mini-Emulsion

A mini-emulsion would be formulated from the materials as described in Table 1 below, although equivalent materials could be substituted.

TABLE 1
Composition Before Polymerization

| Component | Function | Weight |
|---|---|---|
| 2-ethyl hexylacrylate | Monomer/co-stabilizer | 9.21 |
| Dioctyl maleate | Monomer/co-stabilizer | 7.52 |
| Vinyl acetate | Monomer | 30.97 |
| Itaconic acid* | Monomer | 0.10 |
| Polyvinyl alcohol** | Viscosity/wetting | 1.54 |
| | agent | |
| Water | Aqueous Phase | 45.64 |
| Nonionic surfactants: | | |
| Alkylaryl polyether alcohol | Surfactant/emulsifier | 0.54 |
| Nonlylphenoxypoly (ethyleneoxy) | Surfactant/emulsifier | 1.28 |
| alcohol | | |
| t-Butyl hydroperoxide | Initiator | 0.08 |
| Post addition initiator | Monomer scavenger | 0.14 |
| Potassium persulfate | Monomer scavenger | 0.26 |
| Sodium bicarbonate | pH adjustment/ | 0.12 |
| | buffering agent | |
| Ammonium hydroxide (28%) | pH adjustment | 0.21 |
| Hydrooxymethane sulfinic acid, | Reducing agent | 0.13 |
| sodium salt, sodium formaldehyde | | |
| sulfoxylate | | |
| Aminoethylethanolamines*** | Defoamer | 0.80 |
| (optional) | | |
| Benzoate esters, oxydiethyl | Hydrophobe and | 1.46 |
| dibenzoate, ethanol, 2,2-oxybis, | plasticizer | |
| -dibenzoate, propanol, oxybis, | | |
| -dibenzoate | | |

*i.e., methylene succinic acid, 2-methylene-butanedioic acid
**low molecular weight, η
***low molecular weight hydrophobic fatty amides

The procedure for emulsification and polymerization would be as follows:

(1) Prepare the monomer component at standard temperature and pressure conditions, by combining together the monomers, initiators, and hydrophobe/plasticizer components and mixing the components thoroughly with a mechanical mixer.

(2) Prepare the aqueous component at standard temperature and pressure conditions:

Add the polyvinyl alcohol (PVA) polymers into the water; Dissolve the polymers in the water while mixing with a mechanical stirrer, and adjust the temperature to dissolve all of the PVA, until the solution is clear;

Reduce the temperature of the aqueous solution to approximately 20-25° C.; and

Mix the surfactants into the aqueous solution while agitating with a mechanical mixer at approximately 20-25° C., until the solution is clear and while avoiding entrainment of air.

(3) Feed the monomer component into the aqueous component while sonicating or using other dispersion equipment to emulsify the two components/phases.

(4) Continue emulsification until completion, indicated by formation of a mini-emulsion having a dispersed phase comprising particles having an average size between 25 and 100 nm.

(5) Prepare the mini-emulsion for polymerization/synthesis reaction by transferring the emulsion to a kettle or other appropriate reaction vessel, and purge the kettle with nitrogen gas for at a high flow rate for about 30 minutes and then at a reduced flow rate.

(6) Stir the mini-emulsion in the kettle, and initiate heating to reach about 80-85° C. for 30-45 minutes.

(7) Cool the mini-emulsion to a temperature of about 75-77° C. Note the exotherm (to monitor/control the polymerization), and maintain cooling/heating control while continuing to agitate. (Avoid too high of a temperature, which will cause the emulsion viscosity to become too high (e.g., greater than about 2000 cps).)

(8) Maintain the temperature for about 4 hrs until 99% conversion is obtained, as determined by oven percent nonvolatile tests. (Aliquot samples from the kettle placed in convection oven at 105° C. to determine volatile components.)

(9) After monomer conversion is complete (as determined in Step 8 above), raise the temperature of the mini-emulsion to 85° C. for two hours, to react away residual monomer if present.

(10) Adjust the pH of the mini-emulsion as needed, with sodium bicarbonate and ammonium hydroxide.

In an optional formulation, an anti-microbial agent is added to the mini-emulsion. For instance, dry powder chlorhexidine would be dispersed into the mini-emulsion, after step (10), in an amount between 0.5 and 10% by weight.

### EXAMPLE 2

### Efficacy of Hydrophilic Skin Barrier Films in Countering Wound Infection

A study was conducted to assess the effectiveness of the hydrophilic skin barrier material in a Walker-Mason Rat Model for Wound Infection. A polymeric mini-emulsion formulation with chlorhexidine, made as described in Example 1, was applied in liquid form to treat a wound infected with a luminescent bacteria.

Anesthetized rats were placed in a mold designed to expose 20% of the body surface area. The exposed area was immersed in 100° C. water for 10 sec to create a scald wound. The wound was then inoculated with a luminescent bacteria strain of proteous mirabilis. Topical anti-microbial agents were applied to the wounds after one to two hours, some treated by application of the polymeric mini-emulsion formulation and others treated by application of a conventional silver sulfdiazine dressing. The polymeric mini-emulsion formulation dried to a flexible film, having a thickness of less than about 20 microns. The wounds were imaged every 48 hours using photon counting technology and equipment located at the U.S. Army Institute of Surgical Research (Fort Sam Houston, Tex.), to measure changes in bacterial load and distribution. Rats were observed for 21 days, and the outcome measured by mortality.

The results of the test demonstrated that the protective skin barrier film with chlorhexidine successfully countered the infectious proteous mirabilis bacteria that killed the unprotected rat specimens.

Modifications and variations of the methods and devices described herein will be obvious to those skilled in the art from the foregoing detailed description. Such modifications and variations are intended to come within the scope of the appended claims.

We claim:

1. A polymer mini-emulsion for forming biocompatible hydrophilic films for topical application to mammalian skin or soft tissues comprising:
a dispersed phase which comprises particles of a biocompatible hydrophilic polymer and having an average particle size between 50 and 750 nm, the polymer particles comprising a polymer of at least one ethylenically unsaturated monomer;
an aqueous continuous phase in which said dispersed phase is dispersed; and
at least one co-stabilizer effective to stabilize the mini-emulsion.

2. The mini-emulsion of claim 1, further comprising at least one pharmaceutical agent.

3. The mini-emulsion of claim 2, wherein said at least one pharmaceutical agent is in the form of a finely divided solid dispersed directly in the aqueous continuous phase.

4. The mini-emulsion of claim 2, wherein said at least one pharmaceutical agent is selected from the group consisting of antimicrobial agents, anesthetic agents, analgesic agents, hemostatic agents, and combinations thereof.

5. The mini-emulsion of claim 2, wherein said at least one pharmaceutical agent comprises an anti-microbial agent.

6. The mini-emulsion of claim 5, wherein said anti-microbial agent comprises chlorhexidine, a chlohexidine salt, or sodium hypochlorite.

7. The mini-emulsion of claim 2, wherein said at least one pharmaceutical agent is in a predispersion that is dispersed in the aqueous continuous phase.

8. The mini-emulsion of claim 1, wherein the polymer particles have an average particle size between 100 and 500 nm.

9. The mini-emulsion of claim 1, wherein the ethylenically unsaturated monomer is selected from the group consisting of styrene, vinyl acetate, C1 through C4 alkyl acrylate, C1 through C4 alkyl methacrylate, butadiene, vinyl chloride, acrylic acid, methacrylic acid, and mixtures thereof.

10. The mini-emulsion of claim 1, wherein the polymer of at least one ethylenically unsaturated monomer comprises one or more acrylic polymers.

11. The mini-emulsion of claim 1, wherein said co-stabilizer comprises a polymeric co-surfactant.

12. The mini-emulsion of claim 1, further comprising a hydrophobe, a plasticizer, or a combination thereof.

13. The mini-emulsion of claim 1, further comprising a cross-linking agent effective to at least partially cross-link the polymer of at least one ethylenically unsaturated monomer.

14. The mini-emulsion of claim 1, wherein the dispersed phase comprises caprylic acid.

15. A method of forming a polymeric film covering on mammalian skin comprising:
topically applying a liquid mini-emulsion onto an area of mammalian skin to form a wet layer, wherein the liquid mini-emulsion comprises a dispersed phase which comprises particles of a biocompatible hydrophilic polymer and having an average particle size between 50 and 750 nm, the polymer particles comprising a polymer of at least one ethylenically unsaturated monomer, an aqueous continuous phase in which said dispersed phase is dispersed, and at least one co-stabilizer effective to stabilize the mini-emulsion; and
drying the wet layer to form a flexible, continuous film adhered to the area of mammalian skin.

16. The method of claim 15, wherein the area of mammalian skin comprises a skin wound.

17. The method of claim 15, wherein the mammalian skin is human skin.

18. The method of claim 15, wherein the mini-emulsion further comprises a at least one pharmaceutical agent.

19. The method of claim 18, wherein the pharmaceutical agent is selected from the group consisting of anti-microbial agents, anesthetic agents, analgesic agents, hemostatic agents, and combinations thereof.

20. The method of claim 18, wherein the pharmaceutical agent comprises an anti-microbial agent in an amount effective to prevent or treat a bacterial infection in tissues in the skin wound.

21. The method of claim 20, wherein the anti-microbial agent comprises chlorhexidine, a chlohexidine salt, or sodium hypochlorite.

22. The method of claim 18, wherein said at least one pharmaceutical agent is in the form of a finely divided solid dispersed directly in the aqueous continuous phase.

23. The method of claim 18, wherein said at least one pharmaceutical agent is in a predispersion that is dispersed in the aqueous continuous phase.

24. The method of claim 18, wherein said at least one pharmaceutical agent is in a liquid which is dispersed or dissolved in the aqueous continuous phase.

25. The method of claim 15, wherein the polymer particles have an average particle size between 100 and 500 nm.

26. The method of claim 15, wherein the ethylenically unsaturated monomer is selected from the group consisting of styrene, vinyl acetate, C1 through C4 alkyl acrylate, C1 through C4 alkyl methacrylate, butadiene, vinyl chloride, acrylic acid, methacrylic acid, and mixtures thereof.

27. The method of claim 15, wherein the polymer of at least one ethylenically unsaturated monomer comprises one or more acrylic polymers.

28. The method of claim 15, wherein the dispersed phase comprises caprylic acid.

29. The method of claim 15, wherein said co-stabilizer comprises a polymeric co-surfactant.

30. The method of claim 15, wherein the flexible, continuous film has an average thickness of between about 10 and about 25 microns.

31. A biocompatible hydrophilic film for adhering to mammalian skin comprising:
a flexible, continuous film comprised of a biocompatible, hydrophilic polymer network formed from a mini-emulsion, wherein the film is permeable to air and water vapor and adherent to mammalian skin or soft tissue.

32. The biocompatible hydrophilic film of claim 31, further comprising at least one pharmaceutical agent.

33. The biocompatible hydrophilic film of claim 32, wherein the pharmaceutical agent is selected from the group consisting of anti-microbial agents, anesthetic agents, analgesic agents, hemostatic agents, and combinations thereof.

34. The biocompatible hydrophilic film of claim 31, wherein the film has an average thickness between about 10 and about 25 microns.

35. The biocompatible film of claim 31, wherein the wherein the film is substantially transparent.

36. The biocompatible film of claim 35, wherein the film is substantially colorless.

37. The biocompatible film of claim 31, further comprising at least one water-dispersible pigment or dye in an amount effective to color the film.

38. The biocompatible film of claim 31, further comprises a disposable substrate on which the film is maintained until transferred and adhered to the mammalian skin.

39. The biocompatible film of claim 31, further comprising fibers dispersed therein.

40. A kit for use in applying a biocompatible hydrophilic film to mammalian skin, the kit of parts comprising:
a container of a liquid mini-emulsion which comprises a dispersed phase which comprises particles of a biocompatible hydrophilic polymer and having an average particle size between 50 and 750 nm, the polymer particles comprising a polymer of at least one ethylenically unsaturated monomer, an aqueous continuous phase in which said dispersed phase is dispersed, and at least one co-stabilizer effective to stabilize the mini-emulsion; and
an applicator means capable of topically applying a layer of the liquid mini-emulsion onto an area of mammalian skin or soft tissues.

41. The kit of claim 40, wherein the mini-emulsion further comprises at least one pharmaceutical agent.

42. The kit of claim 40, wherein the applicator means comprises a brush, a roller, a collection of woven or non-woven fibers, or a sprayer.

43. A method of covering mammalian skin with an adherent polymeric film comprising:
providing a biocompatible, hydrophilic film which is formed from a liquid mini-emulsion comprising a dispersed phase which comprises particles of a biocompatible hydrophilic polymer and having an average particle size between 50 and 750 nm, the polymer particles comprising a polymer of at least one ethylenically unsaturated monomer, an aqueous continuous phase in which said dispersed phase is dispersed, and at least one co-stabilizer effective to stabilize the mini-emulsion; and
contacting the biocompatible, hydrophilic film to an area of mammalian skin so that the film becomes adhered to the area of mammalian skin.

44. The method of claim 43, wherein the biocompatible hydrophilic film further comprises at least one pharmaceutical agent.

45. The method of claim 43, wherein the biocompatible hydrophilic film has an average thickness between about 10 and about 50 microns.
